(19) United States
(12) Patent Application Publication
(10) Pub. No.: US 2023/0129158 A1
(43) Pub. Date: Apr. 27, 2023

(54) DISINFECTION WAND

(71) Applicant: Phonesoap LLC, Lehi, UT (US)

(72) Inventors: Wesley David LaPorte, Provo, UT (US); Henry Joseph Howell, Herriman, UT (US); Steven James Perry, Lehi, UT (US); Preston Evan Price, Cottonwood Heights, UT (US); Daniel Harrison Barnes, Lehi, UT (US)

(21) Appl. No.: 17/452,355

(22) Filed: Oct. 26, 2021

(51) Int. Cl. A61L 2/10; A61L 2/28

(52) U.S. Cl. CPC A61L 2/10; A61L 2/28; A61L 2202/11; A61L 2202/14

(57) ABSTRACT

A manually operable disinfection wand provides to a user feedback indicating effectiveness of disinfection during treatment of a target surface. The disinfection wand comprises an illuminator section having an illuminator light source, a handle section, a coupling mechanism, and a position sensor. The coupling mechanism enables a user to rotate an illuminator cover in the illuminator section relative to the handle section to selectively allow passing of light emitted by the illuminator light source for incidence on the target surface. The illuminator cover can be rotated relative to the handle section also to block the light emitted by the illuminator light source. The position sensor is placed in the disinfection wand to produce for observation by the user a signal that is indicative of speed of motion of the disinfection wand and thereby its effectiveness in disinfecting the target surface.

### TECHNICAL FIELD

This disclosure relates to a portable, light-based disinfection device. More specifically, this disclosure relates to a manually operable disinfection wand that provides feedback to a user indicating effectiveness of disinfection during treatment of a target surface.

### BACKGROUND

Portable disinfection devices, such as disinfection wands, can offer a quick and easy way of decontaminating an array of surfaces such as from viruses, bacteria, and molds. The devices are typically fitted with portable ultraviolet-c (UVC) light that can inactivate or kill various pathogens. The UVC light, when emitted in sufficient quantity, alter DNA and RNA in pathogens. To disinfect a surface, the light source must be held within a distance from the surface for at least a certain amount of time to effectively treat the surface. The device is gradually moved around the surface to disinfect the area. Since such disinfection devices can damage all DNA and RNA, the devices should be designed or used in a manner that protects the user from damage caused by the light source.

### SUMMARY

A manually operable disinfection wand provides feedback to a user indicating effectiveness of disinfection during treatment of a surface. Portable disinfection devices typically emit a light that can alter DNA and RNA in pathogens upon treatment within a particular distance for a specified period of time. Feedback to the user during usage of a disinfection device can be of benefit to sufficiently treat a target surface. The manually operable disinfection wand described herein comprises an illuminator section, a handle section, a coupling mechanism, and a position sensor. The illuminator section includes an illuminator cover that has an illuminator coupling end, an illuminator window, and an interior region containing a bulb bracket. The bulb bracket has a coupling member and is configured to hold an illuminator light source and support an illuminator light reflector. The handle section includes a housing that has a free end, a handle coupling end, an interior region containing a power supply which provides electrical power to the illuminator light source, and a charging port through which electrical charge is delivered to the power supply for storage and for stimulating light emission from the illuminator light source. The coupling mechanism is positioned between the coupling ends of the illuminator and the handle. The coupling mechanism includes a spinning ring at the illuminator coupling end configured to rotate about the coupling member on the bulb bracket, and at the handle coupling end, the coupling mechanism includes a mechanical ring fixed to the coupling member on the bulb bracket. This coupling mechanism enables the user to rotate the illuminator cover relative to the handle section to selectively align the illuminator window with the illuminator light source to pass the light emitted by the illuminator light source for incidence on a target surface. The illuminator cover can also be rotated relative to the handle section to position the illuminator window away from the light source to block the light emitted by the illuminator light source. The position sensor is placed in the disinfection wand to produce a signal indicative of speed of motion of the disinfection wand, so that the light emitted by the illuminator light source and incident on the target surface is effective to disinfect the target surface.

Additional aspects and advantages will be apparent from the following detailed description of preferred embodiments, which proceeds with reference to the accompanying drawings.

### BRIEF DESCRIPTION OF THE DRAWINGS

FIG. 1 is an oblique view of a disclosed disinfection wand, according to one embodiment of the present disclosure, in a non-illuminating configuration.

FIGS. 2 and 3 are, respectively, bottom and top plan views of the disinfection wand of FIG. 1 in the non-illuminating configuration.

FIG. 4 is a right-side elevation view of the disinfection wand of FIG. 2.

FIG. 5 is a rear end view of the disinfection wand of FIG. 1.

FIG. 6 is an oblique view of a disinfection wand, according to another embodiment of the present disclosure, shown in the non-illuminating configuration with a radiation-protection finger guard in a closed state.

FIG. 7 is an oblique view of the top side of the disinfection wand of FIG. 6 shown in an illuminating configuration with a radiation-protection finger guard in an open state.

FIG. 8 is an oblique view of the left-side of the disinfection wand of FIG. 6 in the illuminating configuration with the radiation-protection finger guard in the open state.

FIG. 9 is an exploded view of an illuminator light source bulb bracket and coupling mechanism components contained in a disinfection wand, according to an embodiment of the present disclosure.

FIG. 10 is a cross-sectional oblique view of coupling mechanism components in a disinfection wand, according to an embodiment of the present disclosure.

FIG. 11 is a fragmentary exploded view of coupling mechanism components of a disinfection wand, according to an embodiment of the present disclosure.

FIG. 12 is a perspective view of an illuminator cover shown in association with a fragmentary cross-sectional view of a coupling mechanism to which the illuminator cover is operatively connected and which connects illuminator and handle sections of a disinfection wand, according to an embodiment of the present disclosure.

FIG. 13 is an isometric view of an illuminator assembly and the coupling mechanism of FIG. 12.

FIG. 14 is an enlarged partly cross-sectional view of the illuminator assembly and the coupling mechanism of FIG. 13.

FIG. 15 is an enlarged fragmentary oblique view of the coupling mechanism shown in FIG. 12.

FIG. 16 is a partly exploded view of internal parts of a disinfection wand, according to an embodiment of the present disclosure.

FIGS. 17 and 18 are cross-sectional oblique views oriented to show different component parts of a disinfection wand, according to an embodiment of the present disclosure.

FIG. 19 is a left-side, fragmentary cross-sectional view of the coupling mechanism of a disinfection wand, according to an embodiment of the present disclosure, in a storage configuration.

FIG. 20 is a left-side, fragmentary cross-sectional view of the coupling mechanism of FIG. 19 in an operating configuration.

FIG. 21 is a cross-sectional view of a spinning ring of the coupling mechanism of FIGS. 19 and 20.

FIG. 22 is a cross-sectional view of a mechanical ring of the coupling mechanism of FIGS. 19 and 20.

### DETAILED DESCRIPTION OF PREFERRED EMBODIMENTS

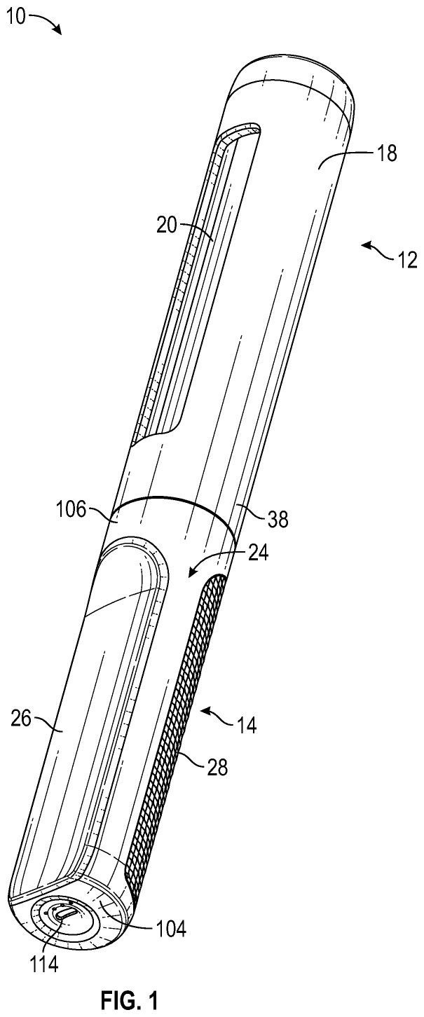

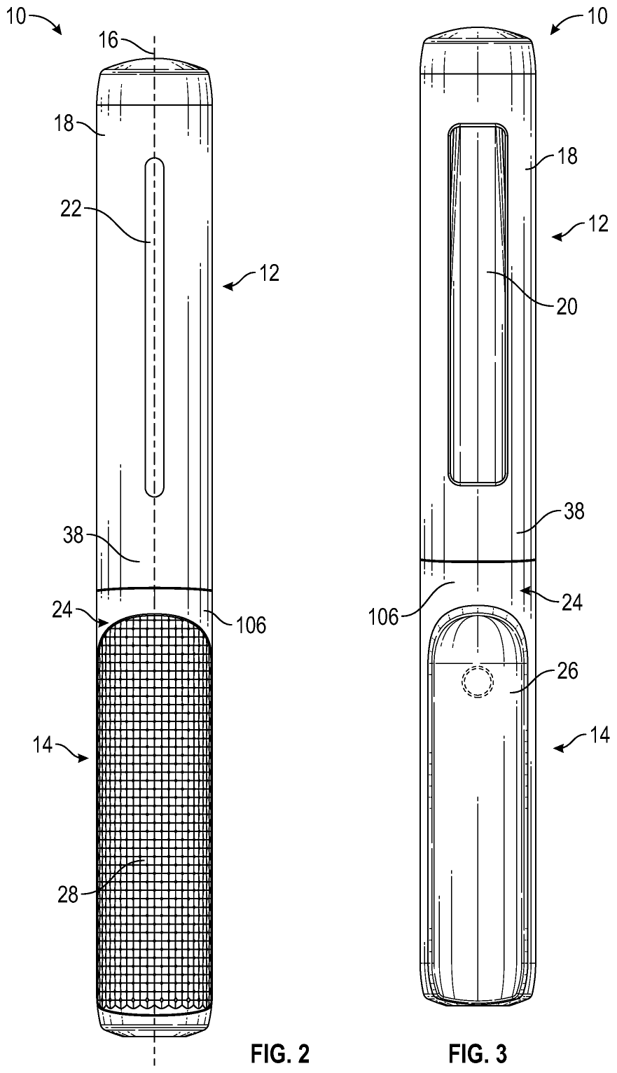

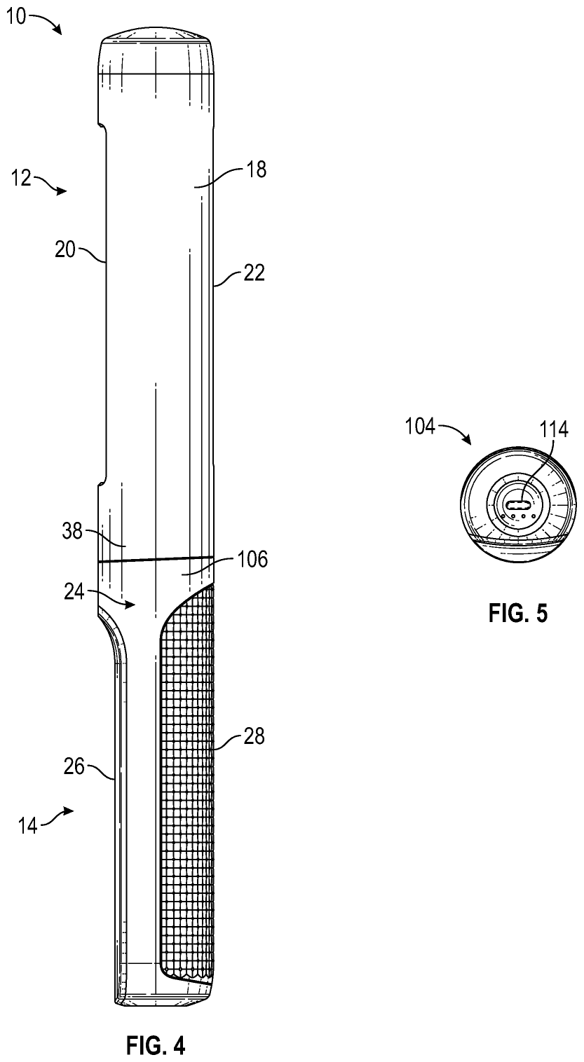

FIGS. 1, 2, 3, 4, 7, and 8 show a disinfection wand 10, according to one embodiment of the present disclosure. The disinfection wand 10 is a portable device that is manually operable to disinfect surfaces. The wand 10 can emit a light that can alter DNA and RNA in pathogens upon treatment within a particular distance for a specified period of time. The wand 10 provides feedback to a user indicating effectiveness of disinfection during treatment of a surface. The manually operable disinfection wand 10 shown includes an illuminator section 12 and a handle section 14 coupled by a coupling mechanism 130. The illuminator section 12 and handle section 14 are coupled to each other in end-to-end alignment.

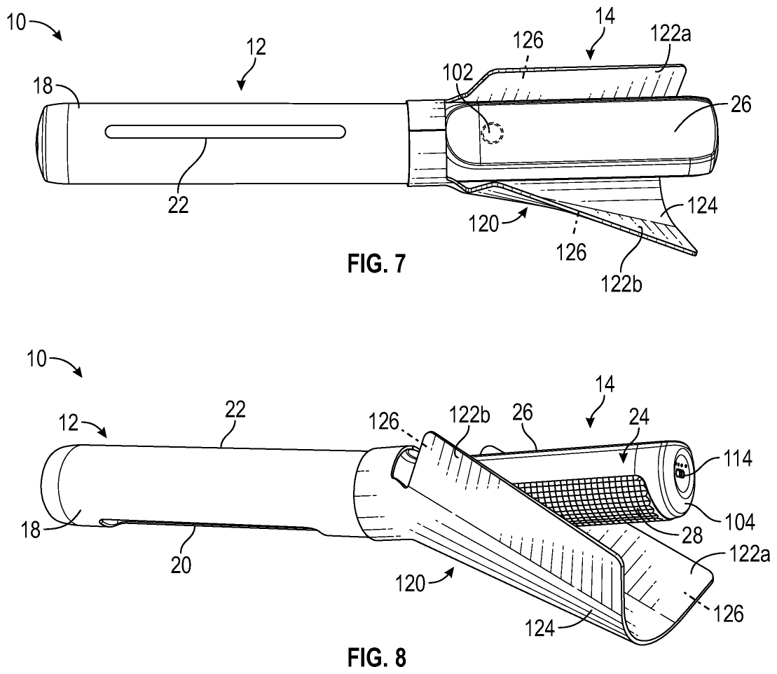

The illuminator section 12 and handle section 14 can be coupled in end-to-end alignment along a central longitudinal axis 16. Illuminator section 12 includes a tubular front cover 18 having an illuminator window 20 and a motion indicator window 22 that are 180° spaced apart from each other. Handle section 14 includes a tubular housing 24 having on one side a recessed region forming a user thumb grip 26 and on an opposite side a textured surface forming a user finger grip 28. Front cover 18 is rotatable about central longitudinal axis 16 relative to handle section 14 so that a user gripping housing 24 between thumb grip 26 and finger grip 28 can turn front cover 18 to selectively set illuminator window 20 to a non-illuminating configuration, shown in FIGS. 1, 2, 3, and 4, and to an illuminating configuration, shown in FIGS. 7 and 8. Motion indicator window 22 is viewable by the user when holding housing 24 with illuminator window 20 in the illuminating configuration.

The following describes in detail the construction and components of illuminator section 12 and handle section 14, and of an internal mechanism coupling them, to achieve the operational performance advantages of disinfection wand 10.

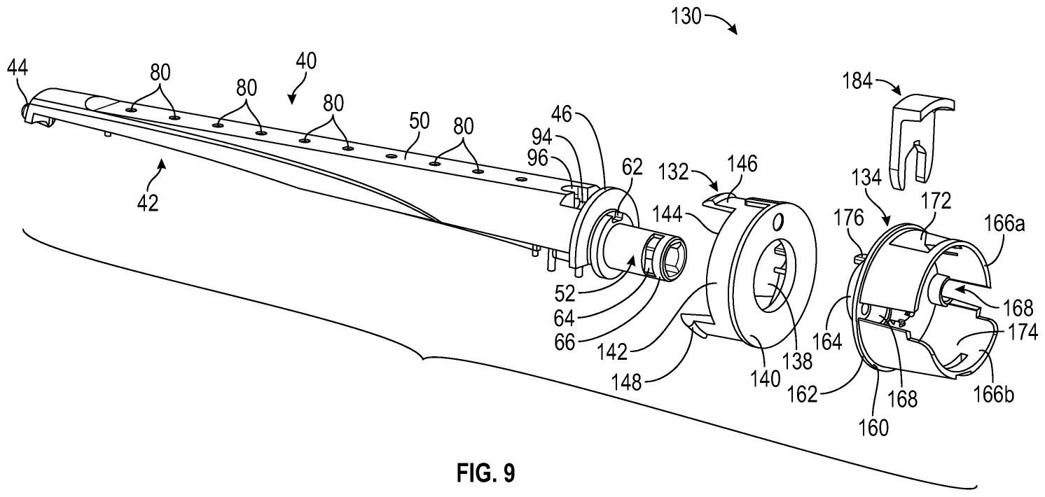

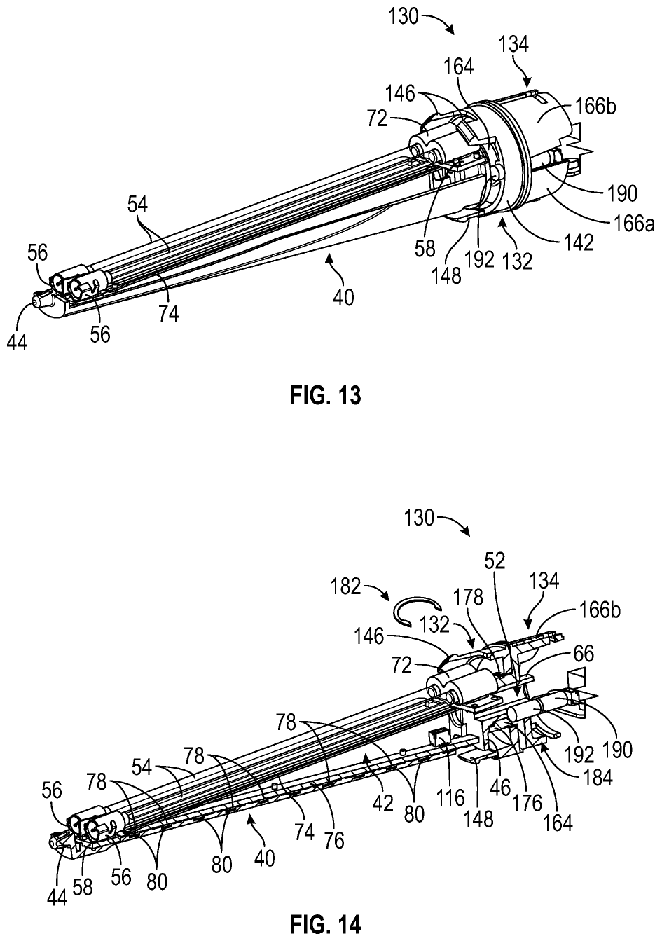

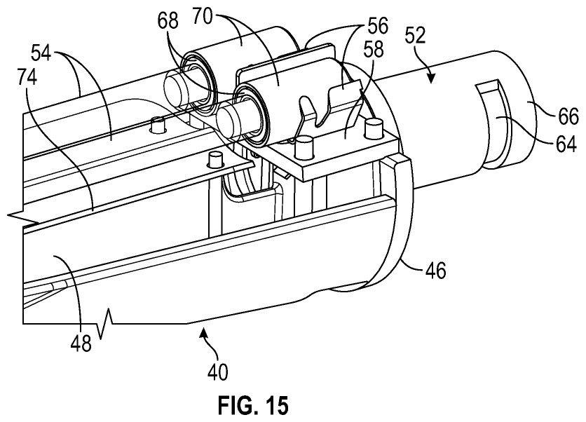

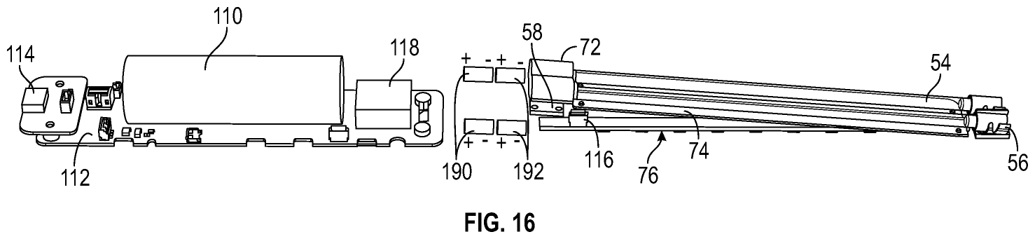

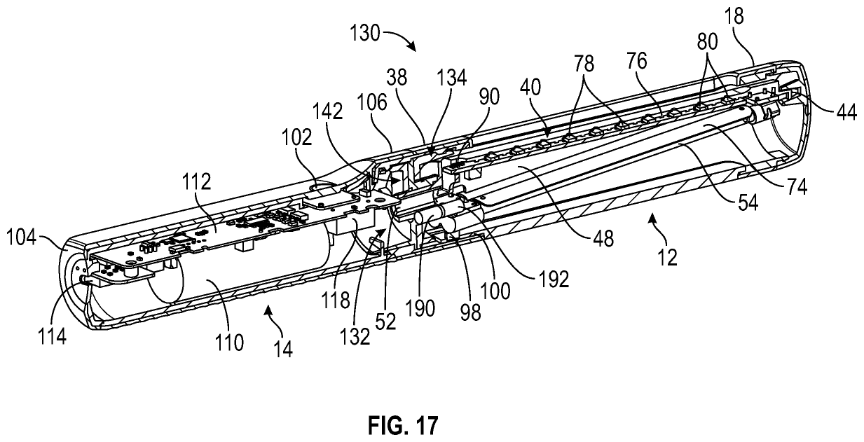

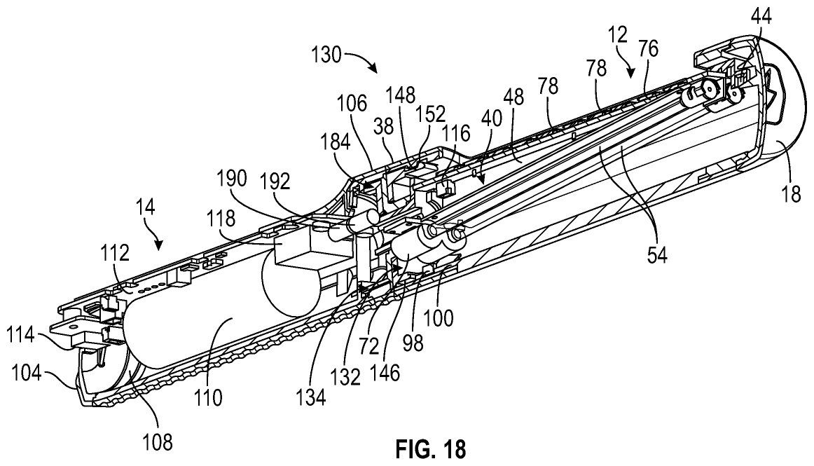

FIGS. 9, 10, 11, 12, 13, 14, 15, 16, 17, and 18 show different views of internal components that are in illuminator section 12. With particular reference to FIGS. 9, 10, 11, 12, 13, 14, and 15, illuminator section 12 comprises an illuminator coupling end 38 and a bulb bracket 40 having along its length a cavity 42 and terminating at a distal end 44 and a proximal end 46. Cavity 42 has an open region 48, a floor 50, and a coupling member 52 at proximal end 46 near illuminator coupling end 38. Bulb bracket 40 holds side-by-side two tubular illuminator light source lamps 54, which form an illuminator light source. A fuse clip 56 holds the free end of each lamp 54 at distal end 44 and the high voltage end of each lamp 54 at proximal end 46 of bulb bracket 40. Separate fuse clip mounts 58 support the two fuse clips 56 at distal end 44 and at proximal end 46 of bulb bracket 40. Illuminator light source 54 is capable of disinfecting a target surface and preferably includes ultraviolet light-emitting cold cathode fluorescent lamp (CCFL) bulbs. An alternative illuminator light source 54 is ultraviolet light-emitting hot cathode fluorescent lamp bulbs. A notch 62 is formed in coupling member 52, near proximal end 46 of bulb bracket 40; and a circumferential recess 64 is formed in coupling member 52, near its distal end 66. Notch 62 and recess 64 are features that contribute to locking bulb bracket 40 and handle section 14 together, as will be described below.

A layer of silicone material 68 placed on the high voltage end of illuminator light source lamp 54 prevent or otherwise limit breakage of lamp 54 upon application of force during lamp installation. A cap 70 sized to fit over silicone material layer 68 covering the high voltage end of each lamp 54 secures it to fuse clip 56 and bulb bracket 40. An endcap umbrella 72 covers cap 70 and separates from each other the adjacent high-voltage ends of lamps 54 at proximal end 46 of bulb bracket 40 to protect disinfection wand 10 from short-circuit malfunction.

An illuminator light reflector 74 positioned between illuminator light source 54 and floor 50 within open region 48 of bulb bracket 40 directs light emitted by illuminator light source 54 toward and outwardly from illuminator window 20 and away from motion indicator window 22 when disinfection wand 10 is in the illuminating configuration. Light reflector 74 is preferably made of aluminum.

A motion indicator light mount 76 in the form of a printed circuit board is heat-staked into floor 50 within open region 48 of bulb bracket 40. Motion indicator light mount 76 holds along its length and applies electrical power to multiple mutually spaced-apart light-emitting devices 78, which collectively form a motion indicator light source. Light-emitting devices 78 are preferably light-emitting diodes (LEDs), ten of which are shown in FIG. 14. Each LED 78 is spatially aligned with an associated hole 80 formed in floor 50 of bulb bracket 40 to allow emission of individual points of light from disinfection wand 10 in the illuminating configuration. Motion indicator light source 78 provides to the user feedback as to whether the user is moving disinfection wand 10 over a target surface at a speed across, an angle relative to, or a distance away from the target surface for effective disinfection treatment. The multiple light-emitting devices 78 can be configured to show an illumination pattern, as described by the following examples, as operational feedback to the user. A first illumination pattern may indicate to the user that disinfection wand 10 has electrical power turned on but is not ready for use for effective disinfection. A second illumination pattern may indicate to the user that disinfection wand 10 is sufficiently powered for use. A third illumination pattern resembling a thermometer code or resulting in a color change may alert the user an extent to which disinfection wand 10 is being moved relative to a target surface at a speed, a distance from, or an angle that is ineffective to sufficiently treat the target surface. Motion indicator light source 78 is positioned so that it emits light sufficiently away from the direction of light emission of illuminator light source 54 to be viewable through motion indicator window 22 to a user treating a target surface with disinfection wand 10.

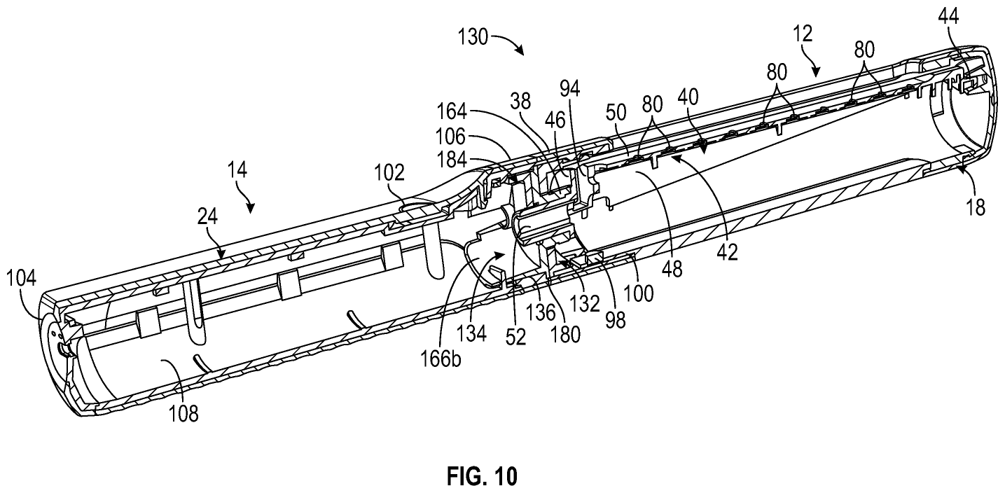

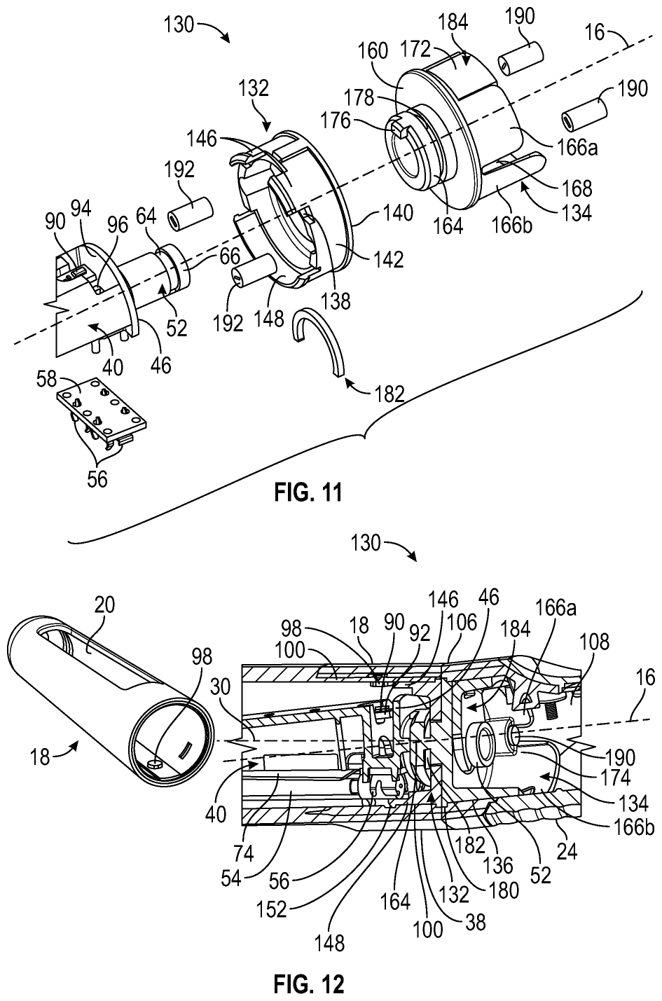

With particular reference to FIGS. 10, 11, and 12, a proximity sensor 90 is set on a sensor mount 92 in a recessed portion 94 of a T-shaped opening 96 in bulb bracket 40, near its proximal end 46. When illuminator section 12 is assembled, bulb bracket 40 fits inside illuminator front cover 18. A front cover magnet 98 is positioned on an internal surface 100 of illuminator front cover 18, opposite from illuminator window 20 and near where illuminator section 12 couples to handle section 14. FIG. 12 shows proximity sensor 90 positioned across from front cover magnet 98, representing when front cover 18 has been rotated to align illuminator window 20 with illuminator light source 54 at the illuminating configuration. Proximity sensor 90 and front cover magnet 98 cooperate to detect when illuminator front cover 18 is at a position where illuminator window 20 is aligned with illuminator light source 54. When it is aligned with front cover magnet 98, proximity sensor 90, which is preferably a Hall effect sensor, senses a maximum strength of a magnetic field generated by front cover magnet 98. The sensing by proximity sensor 90 of a maximum magnetic field is used as a mechanism to enable application of electrical power to disinfection wand 10 or enable emission of light from illuminator light source 54 only when disinfection wand 10 is in the illuminating configuration. Illuminator front cover 18 is configured to rotate relative to handle section 14, and its position serves as a signal indicating sensing by proximity sensor 90 of a maximum magnetic field to allow, or of a weaker magnetic field to prevent, user activation of a power actuator 102 provided on handle section 14 of disinfection wand 10.

FIGS. 12, 16, 17, and 18 show different views of the internal components of handle section 14. Handle section 14 comprises tubular housing 24 having a free end 104, a handle coupling end 106, and an interior region 108. A power supply 110 is mounted on a circuit board 112 that is contained in interior region 108 and operatively connects power supply 110 to illuminator section 12 to provide electrical power to illuminator light source 54. A charging port 114 mounted on circuit board 112 is located preferably at handle free end 104 of disinfection wand 10, as shown in FIGS. 1, 5, and 17, and is connected to power supply 110 to deliver electrical charge for storage and for stimulating light emission from illuminator light source 54. Power supply 110 is charged by connecting a battery charging module to charging port 114. Electrical power from power supply 110 is applied to the high voltage end of illuminator light source 54 and to indicator light source 78 with a wire cable (not shown) to an electrical connector 116. Electrical connector 116 is located preferably on indicator light mount 76 in handle section 14.

A position sensor 118 mounted on circuit board 112, preferably near coupling end 106 of handle section 14, produces a signal that indicates speed of motion, as well as motion varying in distance from a target surface, angular motion and rotational motion, imparted by a user of disinfection wand 10. The signal can indicate to a microcontroller that the disinfection wand 10 may be moving too fast. The microcontroller can, based on the signal, control an external user interface (e.g., produce visual cues on motion indicator light source 78, activate a haptic motor) and thereby provide disinfection efficacy feedback to a user moving disinfection wand 10 over a target surface. The feedback may inform the user that a speed threshold has been surpassed and effective disinfection is no longer occurring. The user does not receive this feedback if the speed threshold has not been surpassed. Position sensor 118, in some embodiments, is preferably an accelerometer that detects multiple axes of linear motion in three-dimensional space, a gyroscope that measures rotational angular velocity in three-dimensional space, a magnetometer (e.g., a 9-axis IMU (Inertial Measurement Unit)), or a combination thereof.

Power actuator 102 is located on circuit board 112 in handle section 14, preferably near handle coupling end 106 and on the same side as user thumb grip 26. Power actuator 102 is configured to allow application of electrical power to illuminator light source 54 after alignment of illuminator window 20 with illuminator light source 54. Application of electrical power to illuminator light source 54 takes place only during user actuation of power actuator 102, whereas electrical power disconnects when the user releases power actuator 102.

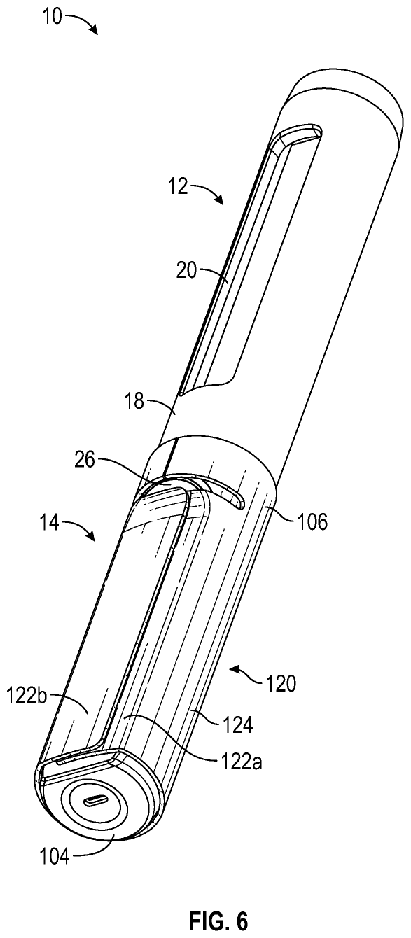

FIG. 6 show finger guard 120 at a closed state obscuring finger grip 28, thumb grip 26, and power actuator 102 of handle section 14 when disinfection wand is not in use. Finger guard 120 may be made of flexible, foldable, or stretchable material and protects, from light emitted by illuminator light source 54, the fingers of a user's hand holding handle section 14. Finger guard 120 is secured to handle section 14 near handle coupling end 106 and has a main body and an opening, such as two partly detached flaps protruding from the main body, on the side of thumb grip 26 for a user to access handle 14 and grip it under finger guard 120. On the side of thumb grip 26, finger guard 120 has two flaps 122a, 122b that can fold over to overlap each other on thumb grip 26, which can result in covering thumb grip 26 and power actuator 102. Flaps 122a and 122b extend from the main body or a guard body 124 of finger guard 120, where guard body 124 is covering the finger grip side of handle 14.

FIGS. 7 and 8 show a finger guard 120 including guard body 124 partly covering finger grip 28 and flaps 122a and 122b open to expose thumb grip 26 of handle section 14 of disinfection wand 10 when it is in use. FIGS. 7 and 8 further illustrate that flaps 122a and 122b extend from the main body or guard body 124 of finger guard 120. Finger guard 120 is partly detached from handle 14 and can open at handle free end 104, as shown in FIGS. 7 and 8. Stated otherwise, finger guard 120 may be configured to detach from and re-attach to the handle 14 at the handle free end 104. One or more magnets 126 may be integrated into or disposed on finger guard 120 to secure it to handle section 14 when the wand 10 is not in use. In another embodiment, one or more magnets 126 may be included in the handle section 14 to attract a ferromagnetic material integrated into or otherwise disposed on the finger guard 120.

Finger guard 120 is attached around wand 10 near handle coupling end 106 and can fold away from handle 14 at a position near handle coupling end 106 on the side of finger grip 28. The folding away of finger guard 120 from handle 14 is allowed due to partially detached finger guard 120 near handle coupling end 106 on the side of thumb grip 26. When finger guard 120 is folded away from handle 14, finger guard 120 is partly detached from handle 14 and open at handle free end 104, allowing a user to access handle 14.

Further protection while operating wand 10 may be provided to the user by the flaps 122a, 122b and guard body 124 covering a user's hand when gripping handle 14 under finger guard 120.

For storage of wand 10, finger guard 120 can fold onto handle 14 from the finger guard's open state to a closed state at handle free end 104 on the finger grip 28 side. In the closed state or closed configuration the flaps 122a, 122b of finger guard 120 can fold over each other against thumb grip 26. The finger guard 120 can remain closed around handle 14, retained, for example, by magnets of the flaps 122a, 122b pulling on a ferromagnetic material in the handle 14 and/or one or more magnets embedded in the handle 14 pulling on a ferromagnetic material of the flaps 122a, 122b and thereby securing the flaps 122a, 122b of finger guard 120 to each other. The one or more magnets maintain the finger guard 120 wrapped around wand 10 and prevent access to power actuator 102.

FIGS. 9, 10, 11, 12, 13, 14, 17, and 18 show different views of a coupling mechanism 130 positioned between illuminator coupling end 38 and handle coupling end 106. Coupling mechanism 130 comprises a spinning ring 132 that fits within front cover 18 and against its internal surface 100 and a mechanical ring 134 that fits within interior region 108 of tubular housing 24 and against its internal surface 136. Coupling mechanism 130 is configured so that spinning ring 132, together with front cover 18, rotates about central longitudinal axis 16 and mechanical ring 134 remains stationary within interior region 108 of tubular housing 24 as a user adjusts the position of illuminator window 20.

Specifically, spinning ring 132 is a modified annulus having a centrally located circular aperture 138 in a planar circular portion 140 and a circumferential side portion 142 with a side margin 144 from which three mutually angularly spaced-apart locking tab segments axially extend. Two shorter length locking tab segments 146 are in face-to-face opposition to a longer length locking tab segment 148. Aperture 138 is sized to receive coupling member 52 as spinning ring 132 is placed over bulb bracket 40 with side margin 144 set adjacent to proximal end 46. When illuminator section 12 is assembled, locking tab segments 146 and 148 snap fit into spatially corresponding step regions 150 and 152, respectively, formed in internal surface 100 of front cover 18.

Mechanical ring 134 is a unitary article formed of a base portion 160 having an annular support surface 162, from which a tubular locking member 164 outwardly extends, and two spaced-apart arcuate side portions 166a and 166b, which axially extend from base portion 160 and are separated from each other by open spaces 168. Side portion 166a has a rectangular opening 172 into a cavity 174 formed in the space between the inner surfaces of side portions 166a and 166b of mechanical ring 134. A locking tab 176 extends outwardly from, and a circumferential groove 178 is formed in, locking member 164. Mechanical ring 134 is positioned within interior region 108 by setting base portion 160 against a recess 180 in internal surface 136 at handle coupling end 106.

Illuminator section 12 and handle section 14 are assembled by setting planar circular portion 140 of spinning ring 132 against annular support surface 162 of mechanical ring 134, with coupling member 52 passing through aperture 138 of spinning ring 132 and tubular locking member 164 of mechanical ring 134, and into cavity 174 of mechanical ring 134. Locking tab 176 of locking member 164 snaps fit into notch 62 in bulb bracket 40; a ring clip 182 fits into circumferential groove 178 in locking member 164 to secure it within spinning ring 132; and a retaining clip 184 passed through opening 172 in cavity 174 in mechanical ring 134 fits into circumferential recess 64 in coupling member 52. This assembly of coupling components holds together illuminator section 12 and handle section 14 while enabling front cover 18, together with spinning ring 132, to rotate about central longitudinal axis 16 and bulb bracket 40 and circuit board 112, together with their associated components, to remain fixed in place.

As shown in an exploded view of the coupling mechanism 130 in FIG. 11, a first set of two spaced-apart magnets 190 is located on mechanical ring 134 at handle coupling end 106 and a second set of two spaced-apart magnets 192 is located on spinning ring 132 at illuminator coupling end 38. Magnets 190 and magnets 192 are spaced apart from magnets of their own set on mechanical ring 134 and spinning ring 132 in a way that magnets 190 and magnets 192 of opposite polarity align when spinning ring 132 is turned 180° relative to mechanical ring 134. Since spinning ring 132 is fixed to front cover 18 and mechanical ring 134 is fixed to handle section 14, and since illuminator window 20 and motion indicator window 22 are spaced apart by 180° from each other on front cover 18, axially aligned magnets 190 and 192 of opposite polarity attract to each other when front cover 18 is in the illuminating configuration and in the non-illuminating configuration. This arrangement of magnets 190 and 192 affords user eye protection by providing the user, without looking directly at disinfection wand 10, haptic sense of locking in the illuminating and non-illuminating configurations when switching between them.

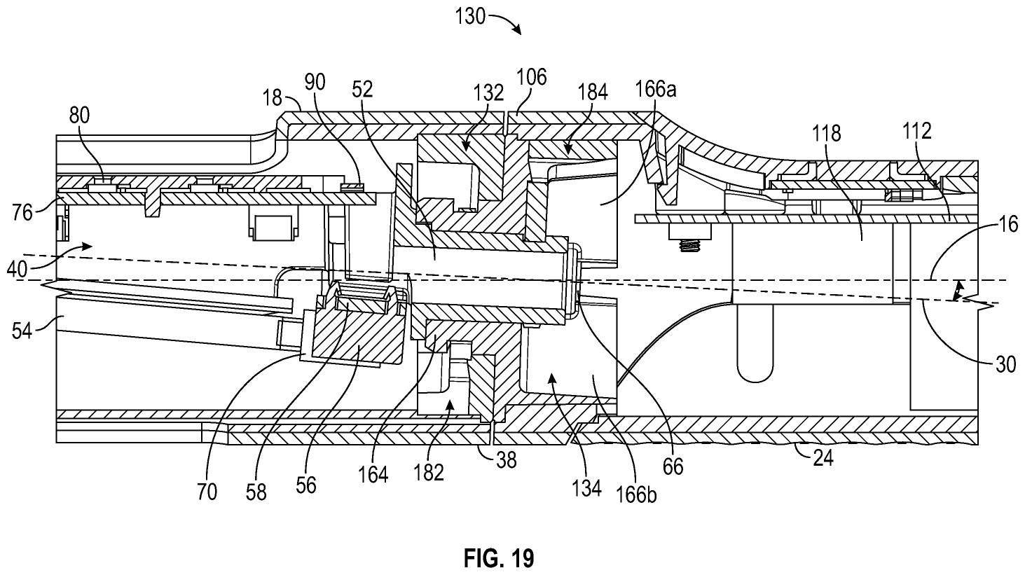

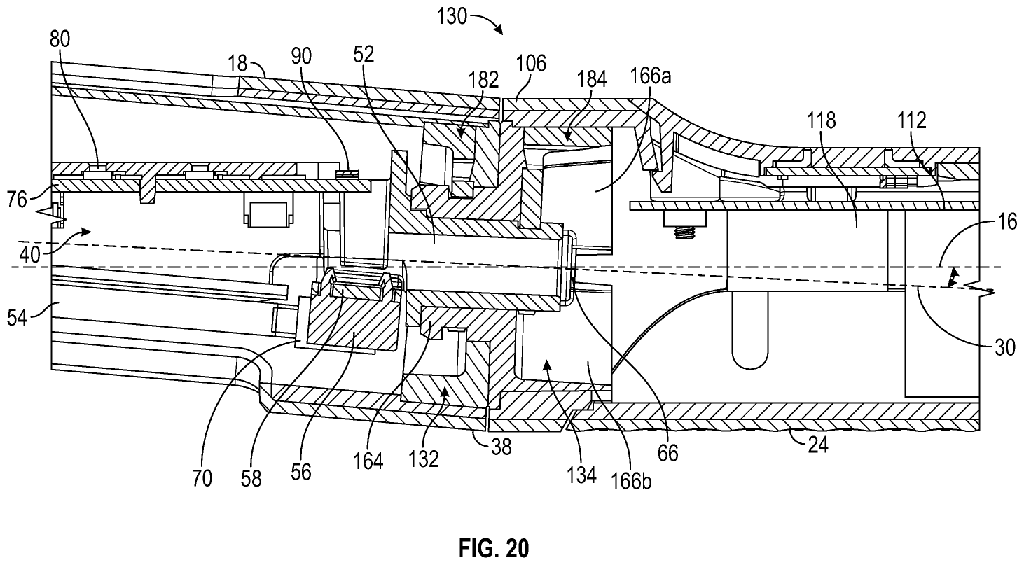

Illuminating configuration of wand 10 can be configured for front cover 18 in illuminating section 12 to lie or otherwise be disposed on a slanted axis compared to handle section 14. The slanted axis of illuminating section 12 compared to handle section 14 may provide more user hand protection from illuminator light source 54. FIGS. 19 and 20 show fragmentary views of the coupling mechanism 130 and front cover 18 in the non-illuminating configuration and the illuminating configuration, respectively. In the non-illuminating configuration, front cover 18 is positioned so that it lies on the same axis as handle 14, namely longitudinal axis 16, as viewed at a side view. Coupling member 52 of bulb bracket 40 is at an angled axis 30, and spinning ring 132 and mechanical ring 134 are configured so that coupling member 52 can be secured at angled axis 30. In the illuminating configuration shown in FIG. 20, front cover 18 has been rotated about bulb 40 relative to handle 14, and the rotation results in front cover 18 lying on angled axis 30 which is offset from longitudinal axis 16 at an angle as viewed at a side view while remaining aligned with longitudinal axis 16 as viewed at top view. At this illuminating configuration, front cover 18 is angled so that light from illuminator light source 54 is directed further away from the user gripping handle 14.

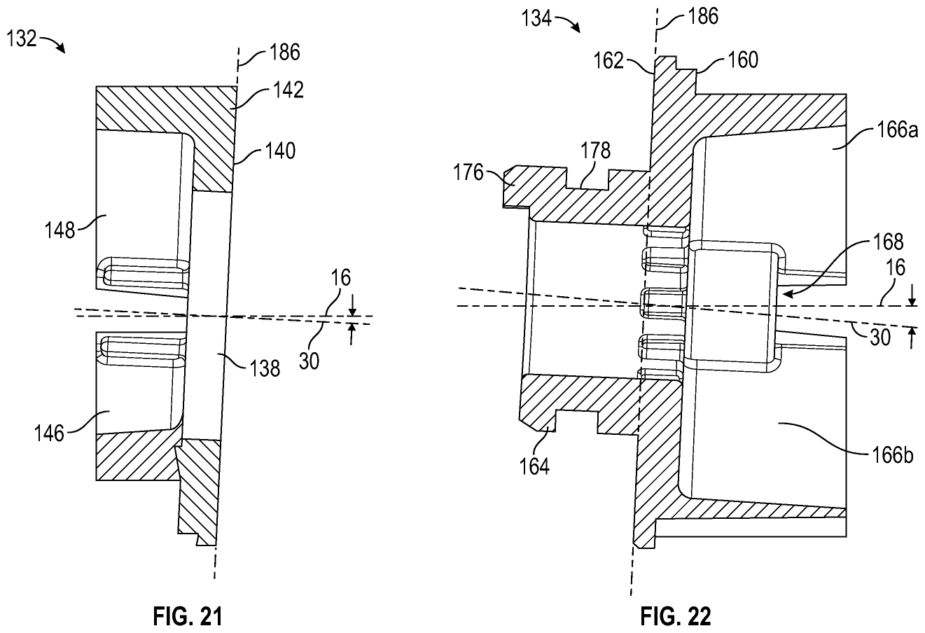

FIGS. 21 and 22 show cross-sectional views of a spinning ring 132 and a mechanical ring 134, respectively, of the coupling mechanism shown in FIGS. 19 and 20. Spinning ring 132 and mechanical ring 134 each have aperture 138 and locking member 164, respectively, through which accept coupling member 52 of bulb bracket 40 at angled axis 30. Annular support surface 162 of mechanical ring 134 and planar circular portion 140 of spinning ring 132 are perpendicular to the angled axis 30 rather than being perpendicular to longitudinal axis of handle 14, so that spinning ring 132 rotates about angled axis 30 on annular support surface 162. A rotational plane 186 is oriented 90° from (i.e., perpendicular to) angled axis 30 and is the plane on which annular support surface 162 and planar circular portion 140 lie. The rotational plane 186 is the plane of rotation on which spinning ring 132 rotates about angled axis 30. Furthermore, spinning ring 132 is fixed to front cover 18 by circumferential side portion 142 which is parallel to longitudinal axis 16. The combination of the relative angles of these components enable that, while planar circular portion 140 is flush against annular support surface 162 when rotated relative to handle 14, the longitudinal axis of front cover 18 can move from being at longitudinal axis 16 to angled axis 30 when wand 10 is at the illuminating configuration (when the illuminator window 20 aligns with illuminator light source 54). Since spinning ring 132 is attached to front cover 18 and mechanical ring 134 is attached to tubular housing 24 of handle 14 as shown in FIGS. 19 and 20, rotating front cover 18 to change from non-illuminating configuration to illuminating configuration of wand 10 results in the change of front cover 18 from lying on longitudinal axis 16 to angled axis 30.

The following is a description of user operation of disinfection wand 10.

Disinfection wand 10 is operated by a user holding in one hand the handle between its finger grip 28 and thumb grip 26 and covering the user's hand with finger guard 120, if provided. If finger guard 120 is provided, the user can access handle 14 by opening finger guard 120 first by its two flaps near thumb grip 26 then sliding the user's hand between handle 14 and finger guard 120. Once the user holds handle 14 in one hand, the user's other hand manually turns front cover 18 180° to align motion indicator window 22 with indicator light source 78. This results in power actuator 102 showing a visible cue to the user and allowing electrical power to be provided to illuminator light source 54 and indicator light source 78 when the user actuates power actuator 102. The turning of front cover 18 also results in front cover 18 transitioning from being disposed on the same axis (e.g., central longitudinal axis 16) as handle 14 to being disposed on an angled axis (e.g., angled axis 30) away from handle 14 when wand 10 is viewed from a side. Front cover 18 is held in such a position due to pairing of magnets 190 and 192 in disinfection wand 10. Upon actuation of the power actuator 102 and application of electrical power to illuminator light source 54 and indicator light source 78, motion indicator light source 78 indicates to the user a level of intensity of light emitted by illuminator light source 54. When motion indicator light source 78 indicates that illuminator light source 54 has reached an effective intensity level for disinfection, shown by all light-emitting devices in indicator light source 78 illuminated along the length of motion indicator window 22, the user can start treating a target surface by scanning the target surface with illuminator light source 54 emitting light onto the target surface.

To sufficiently disinfect a target surface, disinfection wand 10 is held close to the target surface and slowly moved across it with illuminator light source 54 emitting light onto the target surface. Motion indicator light source 78 will continue to emit blue light when disinfection wand 10 is being used in a manner that sufficiently disinfects the target surface. If disinfection wand 10 is moved in a way that is ineffective to disinfect a the target surface, such as when the user rotates disinfection wand 10 so that light emitted by illuminator light source 54 turns away from the target surface or the user moves the disinfection wand 10 at a speed insufficient for disinfection across the target surface, motion indicator light source 78 will emit red light to alert the user of improper use of disinfection wand 10 to disinfect a surface.

To stop disinfecting the target surface, the user releases power actuator 102, after which motion indicator light source 78 indicates to the user that electrical power is not provided to the illuminator light source 54 by lack of light emission by the light devices of motion indicator light source 78. The user can restart using disinfection wand 10 to disinfect a target surface by again actuating power actuator 102. When restarting the disinfection wand 10 between actuations of power actuator 102, the user should ensure motion indicator light source 78 indicates full power level for proper use of disinfection wand 10 to disinfect a target surface.

Powering off disinfection wand 10 to prevent electrical power supplied by actuating power actuator 102 and for storage is accomplished by the user releasing power actuator 102 and turning front cover 18 180° so that motion indicator window 22 is no longer aligned with indicator light source 78 and so that illuminator window 20 is no longer aligned with illuminator light source 54. Magnets 190 and 192 align and pair when front cover 18 is at this position, holding front cover 18 at this non-illuminating configuration.

Power supply 110 in disinfection wand 10 preferably includes a rechargeable battery; a charger module (not shown) connected to charging port 114 delivers electrical charge to power supply 110.

It will be obvious to those having skill in the art that many changes may be made to the details of the above-described embodiments without departing from the underlying principles of the invention. The scope of the present invention should, therefore, be determined only by the following claims.

1. A manually operable disinfection wand comprising:
an illuminator section including an illuminator light source to be actuated to produce disinfecting light to be directed at a surface to be disinfected;
a handle to be grasped by a user to manually operate the disinfection wand, the handle including a power actuator to actuate the illuminator source, the handle having a handle free end and a handle coupling end, wherein the handle is operatively coupled to the illuminator section at the handle coupling end; and
a finger guard fixedly attached to the handle at the handle coupling end and configured to releasably engage the handle at the handle free end, the finger guard comprising:
a guard body configured to at least partially encompass the handle; and
a first flap and a second flap each extending from the guard body, each to releasably couple together in a closed configuration to prevent activation of the power actuator on the handle and to detach to transition to an open configuration to expose the power actuator,
wherein, in the open configuration, the guard body is positioned to shield a hand of a user grasping the handle from light produced by the illuminator section during operation.

2. The manually operable disinfection wand of claim 1, wherein one or both of the first flap and the second flap include a magnetic material to attract to a ferromagnetic material to draw the first flap and second flap into releasable coupling together and thereby to the closed configuration.

3. The manually operable disinfection wand of claim 2, wherein the ferromagnetic material is disposed in the other of the first flap and the second flap including the magnetic material.

4. The manually operable disinfection wand of claim 2, wherein the ferromagnetic material is disposed in the handle.

5. The manually operable disinfection wand of claim 1, wherein one or both of the first flap and the second flap include a ferromagnetic material and the handle further comprises a magnet to attract to the ferromagnetic material to draw the first flap and second flap into releasable coupling together into the closed configuration.

6. The manually operable disinfection wand of claim 1, wherein one or both of the first flap and the second flap include a magnet and the handle further comprises a ferromagnetic material, wherein the magnet tends to be drawn to the ferromagnetic material to draw the first flap and second flap into releasable coupling together in the closed configuration.

7. The manually operable disinfection wand of claim 1, wherein one or the first flap includes a first magnet and the second flap includes a second magnet, wherein the first magnet and the second magnet tend to be drawn together into releasable coupling to dispose the finger guard to the closed configuration.

8. The manually operable disinfection wand of claim 1, wherein the power actuator is positioned on the handle section and configured to activate application of the electrical power to the illuminator light source after alignment of an illuminator window of the illuminator section with the illuminator light source.

9. The manually operable disinfection wand of claim 1, wherein the first flap and the second flap in the closed configuration fold together and against the handle.

10. A disinfection wand comprising:
an illuminator section including an ultraviolet cathode bulb to disinfect a surface;
a handle section to be grasped by a user to manually operate the disinfection wand, wherein the handle section is operatively coupled to the illuminator section; and
a finger guard attached to the handle section, the finger guard comprising:
a guard body configured to at least partially cover the handle section; and
a first flap and a second flap each extending from the guard body, each flap to releasably engage together in a closed configuration to cover the handle section and to disengage to transition to an open configuration to expose the handle section to be grasped by the user,
wherein, in the open configuration, the guard body is positioned to limit exposure of a hand of a user grasping the handle from light emitted by the ultraviolet cathode bulb during operation of the disinfection wand.

11. The disinfection wand of claim 10, wherein one or both of the first flap and the second flap include a magnetic material to attract to a ferromagnetic material to draw the first flap and second flap into releasably engage together and thereby to the closed configuration.

12. The disinfection wand of claim 11, wherein the ferromagnetic material is disposed in the other of the first flap and the second flap including the magnetic material.

13. The disinfection wand of claim 11, wherein the ferromagnetic material is disposed in the handle section.

14. The disinfection wand of claim 10, wherein one or both of the first flap and the second flap include a ferromagnetic material and the handle section further comprises a magnet to attract to the ferromagnetic material to draw the first flap and second flap into releasably engage together into the closed configuration.

15. The disinfection wand of claim 10, wherein one or both of the first flap and the second flap include a magnet and the handle section further comprises a ferromagnetic material, wherein the magnet tends to be drawn to the ferromagnetic material to draw the first flap and second flap into releasably engage together in the closed configuration.

16. The disinfection wand of claim 10, wherein one or the first flap includes a first magnet and the second flap includes a second magnet, wherein the first magnet and the second magnet tend to be drawn together to releasably engage to dispose the finger guard to the closed configuration.

17. The disinfection wand of claim 10, wherein a power actuator is positioned on the handle section and configured to activate application of electrical power to the ultraviolet cathode bulb after alignment of an illuminator window of the illuminator section with the ultraviolet cathode bulb.

18. The disinfection wand of claim 10, wherein the first flap and the second flap in the closed configuration fold together and against the handle section.

19-41. (canceled)

42. A manually operable disinfection wand formed of multiple sections coupled in coaxial alignment along a central longitudinal axis, comprising:
an illuminator section including a tubular illuminator cover having an illuminator coupling end and an illuminator window, the illuminator cover having an interior region in which is contained a bulb bracket having a proximal end portion and a distal end portion and defining along a length of the bulb bracket a cavity having an open region, a floor, and a coupling member at the proximal end portion, the bulb bracket configured to hold at the proximal end portion and the distal end portion, respectively, a high voltage end and an opposite end of a tubular illuminator light source, and to support an illuminator light reflector in a position to reflect, in a direction away from the floor and out of the cavity, light emitted by the illuminator light source;
a handle section including a tubular housing having a handle free end and a handle coupling end, the housing having an interior region in which a power supply is contained and operatively connected to the illuminator section to provide electrical power to the illuminator light source, and in which a charging port is operatively connected to the power supply to deliver electrical charge for storage and for stimulating light emission from the illuminator light source;
a coupling mechanism positioned between the illuminator coupling end and the handle coupling end, the coupling mechanism including, at the illuminator coupling end, a spinning ring configured for rotation about the coupling member at the proximal end portion of the bulb bracket and, at the handle coupling end, a mechanical ring fixed to the coupling member at the proximal end portion of the bulb bracket, thereby enabling user rotation of the illuminator cover relative to the handle section to selectively align the illuminator window with the illuminator light source to pass the light emitted by the illuminator light source for incidence on a target surface or position the illuminator window away from the light source to block the light emitted by the illuminator light source; and
a position sensor placed to produce a signal indicative of speed of motion of the disinfection wand so that the light emitted by the illuminator light source and incident on the target surface is effective to disinfect it.

43. The manually operable disinfection wand of claim 42, in which the tubular illuminator cover has an indicator window angularly displaced from the illuminator window about the central longitudinal axis, and further comprising:
an indicator light source mount positioned on the bulb bracket at a location between the illuminator light reflector and the floor and configured to support and apply the electrical power to an indicator light source, light emitted by the indicator light source being directed away from the light emitted by the illuminator light source and viewable through the indicator window during disinfection of the target surface.

44-54. (canceled)
